(19) United States
(12) Patent Application Publication
(10) Pub. No.: US 2016/0331582 A1
(43) Pub. Date: Nov. 17, 2016

(54) HELMET HAVING AN EMBEDDED COOLING ARRAY

(71) Applicant: INTERNATIONAL BUSINESS MACHINES CORPORATION, Armonk, NY (US)

(72) Inventors: JAMES R. KOZLOSKI, NEW FAIRFIELD, CT (US); MARK C. H. LAMOREY, WILLISTON, VT (US); CLIFFORD A. PICKOVER, YORKTOWN HEIGHTS, NY (US); JOHN J. RICE, MOHEGAN LAKE, NY (US)

(21) Appl. No.: 14/745,495

(22) Filed: Jun. 22, 2015

Related U.S. Application Data
(63) Continuation of application No. 14/709,568, filed on May 12, 2015.

(51) Int. Cl. A61F 7/10; A61F 7/00; A42B 3/04; A42B 3/12; A42B 3/28

(52) U.S. Cl. CPC A61F 7/10; A42B 3/125; A42B 3/285; A42B 3/046; A61F 7/007; A61F 2007/0002; A61F 2007/0056; A61F 2007/0086; A61F 2007/0096

(57) ABSTRACT

Embodiments include methods for cooling a helmet having an embedded cooling array with an external cooling system. Aspects include determining that the helmet has experienced a severe impact, wherein the helmet includes a padding configured to protect a user of the helmet from impacts and connecting the external cooling system to the embedded cooling array of the helmet. Aspects further include activating the external cooling system, wherein activating the external cooling system decreases an internal temperature of the helmet via the embedded cooling array.

### DOMESTIC PRIORITY

This application is a continuation of U.S. application Ser. No.: 14/709,568; Attorney Docket: YOR920150167US1; Filed: May 12, 2015; which is related to U.S. U.S. application Ser. No.: 14/709,575; Attorney Docket: YOR920150161US1; Filed: May 12, 2015; application Ser. No.: 14/709,574; Attorney Docket.: YOR92015162US1; Filed May 12, 2015; U.S. application Ser. No.: 14/709,572; Attorney Docket: YOR920150163US1; Filed: May 12, 2015; U.S. application Ser. No.: 14/709,570; Attorney Docket: YOR920150164US1; Filed: May 12, 2015; U.S. application Ser. No.: 14/709,563; Attorney Docket: YOR920150165US1; Filed: May 12, 2015; U.S. application Ser. No.: 14/709,564; Attorney Docket YOR920150168US1; Filed: May 12, 2015; U.S. application Ser. No.: 14/664,987; Filed March 23, 2015; Attorney Docket: YOR920150038US1; U.S. application Ser. No.: 14/664,989; Filed: Mar. 23, 2015; Attorney Docket: YOR920150039US1; and U.S. application Ser. No.: 14/664,991; Filed: March 23, 2015; Attorney Docket: YOR920150040US1, the contents of each of which are herein incorporated by reference in their entirety.

### BACKGROUND

The present disclosure relates to a helmet having an embedded cooling array, and more specifically, to a helmet having an embedded cooling array for coupling to an external cooling system following a potential traumatic brain injury.

Generally speaking, safety is a primary concern for both users of helmets and manufacturers of helmets. Helmets are used by individuals that participate in activities that have risk of head trauma, such as the area of sports, biking, motorcycling, etc. While helmets have traditionally been used to provide protection from blunt force trauma to the head, an increased awareness of concussion causing forces has motivated a need for advances in helmet technology to provide increased protection against concussions. A concussion is a type of traumatic brain injury that is caused by a blow to the head that shakes the brain inside the skull due to linear or rotational accelerations. Recently, research has linked concussions to a range of health problems, from depression to Alzheimer's, along with a range of brain injuries. Unlike severe traumatic brain injuries, which result in lesions or bleeding inside the brain and are detectable using standard medical imaging, a concussion is often invisible in brain tissue, and therefore only detectable by means of a cognitive change, where that change is measurable by changes to brain tissue actions, either neurophysiological or through muscle actions caused by the brain and the muscles resulting effects on the environment, for example, speech sounds.

Recent research suggests that hypothermia, or cooling of the head, can be used as an effective treatment for individuals that have suffered a traumatic brain injury. In some cases, such as with football players, an individual wearing a helmet may suffer an impact that causes a traumatic brain injury and the helmet may need to be removed to effectively cool the head of the individual. However, due to safety concerns, the removal of the helmet by untrained personnel is not desirable. Accordingly, the ability to quickly perform cooling the head of the individual is inhibited by the helmet.

### SUMMARY

In accordance with an embodiment, a method for cooling a helmet having an embedded cooling array with an external cooling system is provided. The method includes determining that the helmet has experienced a severe impact, wherein the helmet includes a padding configured to protect a user of the helmet from impacts and connecting the external cooling system to the embedded cooling array of the helmet. The method further includes activating the external cooling system, wherein activating the external cooling system decreases an internal temperature of the helmet via the embedded cooling array.

In accordance with another embodiment, a system for treating traumatic brain injuries in individuals wearing a helmet is provided. The system includes a helmet having a padding configured to protect a user of the helmet from impacts, an embedded cooling array and one or more cooling system ports coupled to the embedded cooling array. The system also includes an external cooling system configured to be coupled to the embedded cooling array via the one or more cooling system ports.

### BRIEF DESCRIPTION OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a block diagram illustrating one example of a processing system for practice of the teachings herein;

FIG. 2 is a block diagram illustrating a helmet having an embedded cooling array in accordance with an exemplary embodiment;

FIG. 3 is a block diagram illustrating a cooling system coupled to a helmet having an embedded cooling array in accordance with an exemplary embodiment;

FIG. 4 is a flow diagram of a method for determining if a helmet has experienced a severe impact accordance with an exemplary embodiment;

FIG. 5 is a block diagram illustrating a system for monitoring a helmet having an embedded cooling array in accordance with an exemplary embodiment; and

FIG. 6 is a flow diagram of a method for cooling a helmet having an embedded cooling array with an external cooling system accordance with an exemplary embodiment.

### DETAILED DESCRIPTION

In accordance with exemplary embodiments, methods and systems for cooling a helmet having an embedded cooling array with an external cooling system are provided. The helmet includes padding that is designed to protect the user against impacts, such as blunt force trauma, to the head. For example, the helmet may be a football helmet, a motorcycle helmet or the like. In exemplary embodiments, the embedded cooling array of the helmet is disposed within and/or in-between the padding on the inside of the helmet. The cooling array is configured to be coupled to an external cooling system that is used to activate the cooling array. In exemplary embodiments, the cooling provided by the cooling array may be controlled by the external cooling system and/or the helmet. For example, the amount and location of the cooling may be controlled by the cooling system and/or the helmet. Accordingly, when a user of a helmet suffers a potential traumatic brain injury, the cooling system can be coupled to the helmet and the user's head can be cooled without having to remove the helmet.

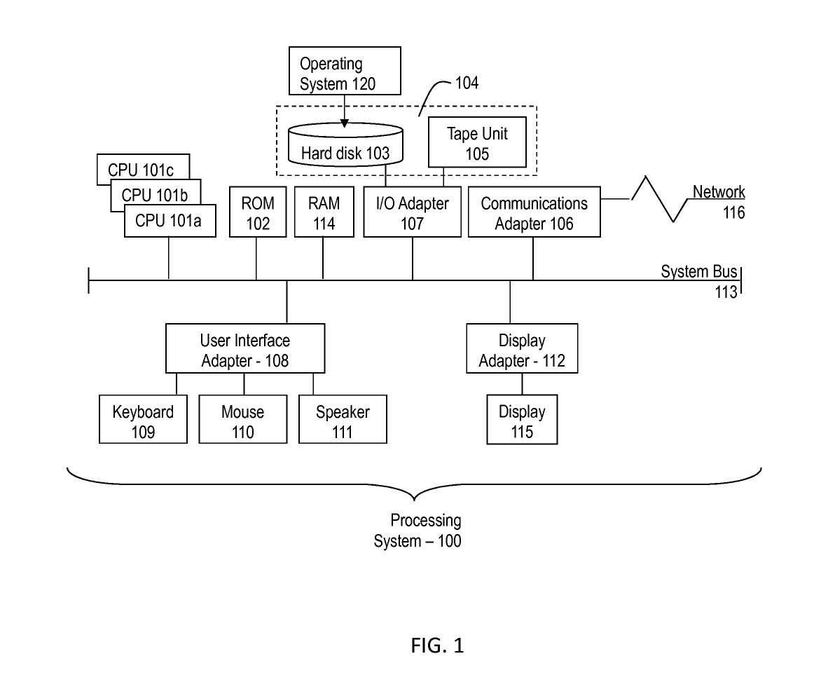

Referring to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 may include a reduced instruction set computer (RISC) microprocessor. Processors 101 are coupled to system memory 114 and various other components via a system bus 113. Read only memory (ROM) 102 is coupled to the system bus 113 and may include a basic input/output system (BIOS), which controls certain basic functions of system 100.

FIG. 1 further depicts an input/output (I/O) adapter 107 and a network adapter 106 coupled to the system bus 113. I/O adapter 107 may be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 may be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which may include a graphics adapter to improve the performance of graphics intensive applications and a video controller. In one embodiment, adapters 107, 106, and 112 may be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which may include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

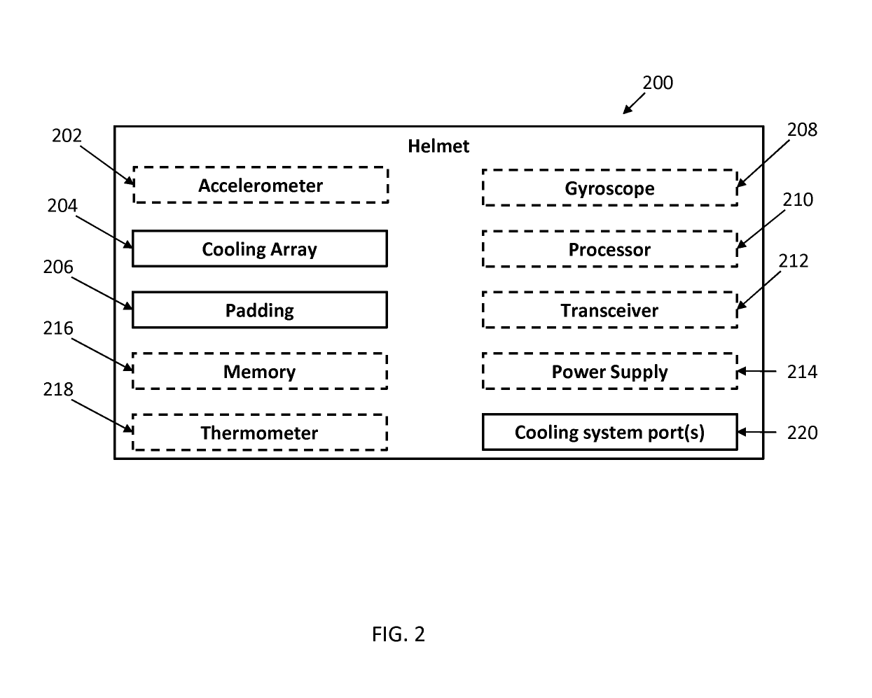

Referring to FIG. 2, a block diagram illustrating a helmet 200 having an embedded cooling array 204 in accordance with an exemplary embodiment is shown. The helmet 200 includes padding 206 that is configured to protect the user against blunt force trauma to the head. The helmet 200 also includes one or more cooling system ports 220 that are configured to connect the cooling array 204 to an external cooling system. In exemplary embodiments, the embedded cooling array 204 of the helmet is disposed within and/or in-between the padding 206 on the inside of the helmet.

In one embodiment, the helmet 200 is a football helmet that includes padding 206 disposed within the interior of the helmet and the cooling array 204 includes a series of tubes for circulating a cooling fluid. The series of tubes may be disposed in-between the padding, within the padding, or any suitable combination of the two. The cooling system ports 220 include one or more ports that are accessible when the helmet is on the user and which connect to the cooling array 204. For example, the helmet may include two ports, an input for receiving cooling fluid and an output for returning cooling fluid to the external cooling system. In exemplary embodiments, there may be several cooling system ports 220 that can be used to selectively provide cooling fluid to only desired areas within the helmet 200.

In exemplary embodiments, the helmet 200 may also include one or more of the following: an accelerometer 202, a gyroscope 208, a processor 210, a transceiver 212, a power supply 214, a memory 216 and a thermometer 218. In exemplary embodiments, the power supply 214 may be a battery configured to provide power to one or more of the accelerometer 202, the gyroscope 208, the processor 210 and the transceiver 212. In exemplary embodiments, the transceiver 212 may be configured to communicate with an external processing system and/or an external cooling system. In addition, the processor 210 may be configured to monitor the thermometer 218 and to provide the internal temperature of the helmet received from the thermometer 218 to the external processing system and/or an external cooling system.

In one embodiment, the processor 210 is configured to receive an output from one or more of the accelerometer 202 and the gyroscope 208 and to determine if a severe impact has occurred. As used herein, the term “severe impact” refers to an impact that is likely to cause a traumatic brain injury. The processor 210 may determine if an impact is severe based on the acceleration and rotation experienced by the helmet. Upon determining that a severe impact has occurred, the processor 210 may create an alert indicating that a severe impact has occurred. Such an alert may be a visual indicator on the helmet or it may be an electronic signal sent via the transceiver 212 to a separate processing system. In exemplary embodiments, the alert can be used to notify personnel that of the need for coupling the external cooling system to the helmet.

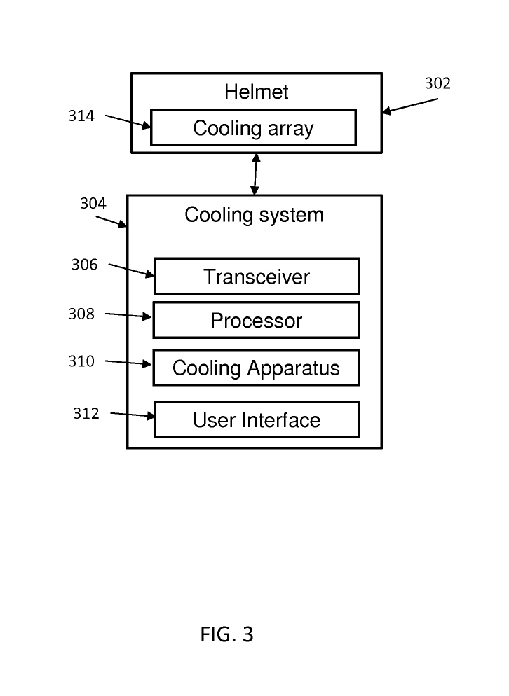

Referring to FIG. 3, a block diagram illustrating a cooling system 304 coupled to a helmet 302 having an embedded cooling array 314 in accordance with an exemplary embodiment is shown. In exemplary embodiments, the cooling system 304 includes a transceiver 306, a processor 308, a cooling apparatus 310 and a user interface 312. The transceiver 306 may be configured to communicate with the transceiver of the helmet and to receive information from the helmet, such as the internal temperature of the helmet. The processor 308 is configured to operate the cooling apparatus 310 based on inputs received from the user interface 312 and from the helmet 302. For example, the user interface 312 may be configured to receive a target temperature from a user and the processor 308 may operate the cooling system 304 to achieve the desired temperature inside the helmet 302. In other embodiments, the user interface 312 may provide manual controls for activating the cooling apparatus 310 and controlling the operation of the cooling apparatus. In exemplary embodiments, an individual may perform an assessment of the cognitive function of the user of the helmet 302 and may ascertain the nature and amount of cooling needed, along with head regions that may benefit most from the cooling. The individual may then use the user interface 312 to control the nature and amount of cooling provided by the cooling system 304 and the cooling array 314.

In exemplary embodiments, the processor 308 of the helmet 302 is configured to perform an assessment of cognitive function to judge the nature and amount of cooling needed. In addition, the assessment may provide an indication of the head regions that may benefit most from the cooling. In one embodiment, the helmet 302 may be able to control the cooling of the head without input from a healthcare professional. In another embodiment, the helmet 302 may record the nature and location of a blow to the head and provide that information to a healthcare professional, who responsively instructs the helmet 302 to apply cooling various regions of the head.

In exemplary embodiments, the cooling array 314 of the helmet 302 and the cooling apparatus 310 of the cooling system 304 may utilize various known techniques to cool the inside of the helmet. For example, the cooling apparatus 310 may be a system for cooling a fluid and pumping the cooled fluid through the cooling array 314, which may include one or more tubes disposed inside the helmet. In another example, the cooling array 314 may include a plurality of thermoelectric cooling pads disposed within the helmet and the cooling apparatus 310 may be an electrical system designed to selectively activate the thermoelectric cooling pads.

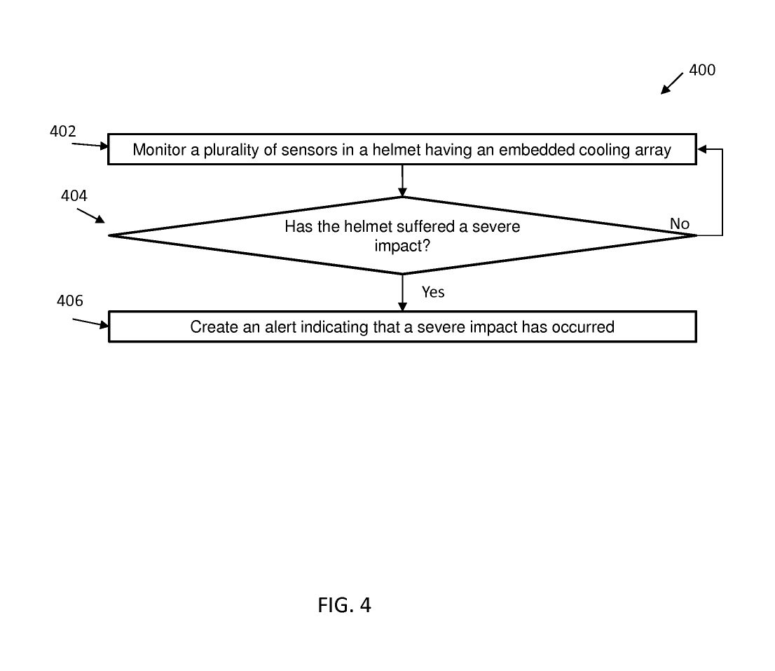

Referring now to FIG. 4, a flow diagram of a method 400 for determining if a helmet has experienced a severe impact in accordance with an exemplary embodiment is shown. As shown at block 402, the method 400 includes monitoring a plurality of sensors in the helmet. In exemplary embodiments, the plurality of sensors includes one or more of an accelerometer and a gyroscope. Next, as shown at decision block 404, the method includes determining if the helmet suffered a severe impact. In exemplary embodiments, determining if the helmet suffered a severe impact includes determining if the acceleration or rotation experienced by the helmet, or a combination thereof, exceeds threshold levels. If the helmet has suffered a severe impact, the method proceeds to block 406 and includes creating an alert indicating that a severe impact has occurred. In one embodiment, the alert may include a visual alert disposed on the helmet such that the user or other personnel can easily identify the alert. In another embodiment, the method may also include transmitting the alert a separate processing system. For example, the helmet may transmit an alert to a processing system on a sideline that indicates that the helmet has suffered a severe impact. The alert may include an identification of the user and the nature of impact, i.e., the amount of detected acceleration and/or rotation. Based on determining that the helmet has not suffered a severe impact, the method 400 continues monitoring the plurality of sensors in the helmet, as shown at block 402.

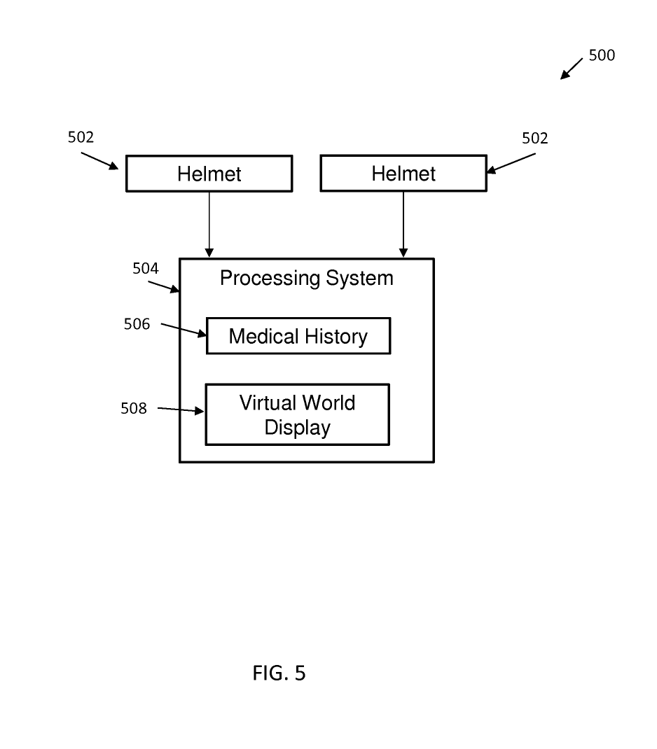

Referring now to FIG. 5, a block diagram illustrating a system 500 for monitoring helmets in accordance with an exemplary embodiment is shown. As illustrated, the system 500 includes one or more helmets 502, such as the one shown and described above with reference to FIG. 2, and a processing system 504, such as the one shown and described above with reference to FIG. 1. The processing system 504 is configured to communicate with the helmets 502 and is also configured to store the medical history 506 of the users of the helmets 502. In exemplary embodiments, the medical history 506 of the users of the helmets 502 may be used by the helmet 502 in determining one or more thresholds used for determining if an impact experienced is severe. In addition, the processing system 504 may include a virtual world display 508 that is configured to provide a display a real-time status of each of the users of the helmets.

In exemplary embodiments, the user's history of collision or medical concerns may be used to determine a traumatic brain injury risk assessment, either by the embedded processor of the helmet or the separate processing system. In exemplary embodiments, an aggregate indication may be used to summarize an overall state of a group of players. This may also help to potentially identify area of risk in the dynamics of player-player interaction, overly aggressive players, playing field conditions, etc. In exemplary embodiments, an automatic feed from a user's history of collision or medical concerns may also be provided to a processor of the helmet in order to update an impact risk model for each category of play. In addition, the processing system 504 may receive a real-time feed of the user's cognitive state, which can be used to update the risk models used by the helmets. The risk models may also be sent to the virtual world display 508 of the game and players, which allows the sports staff health professionals to visualize the nature of potential problems.

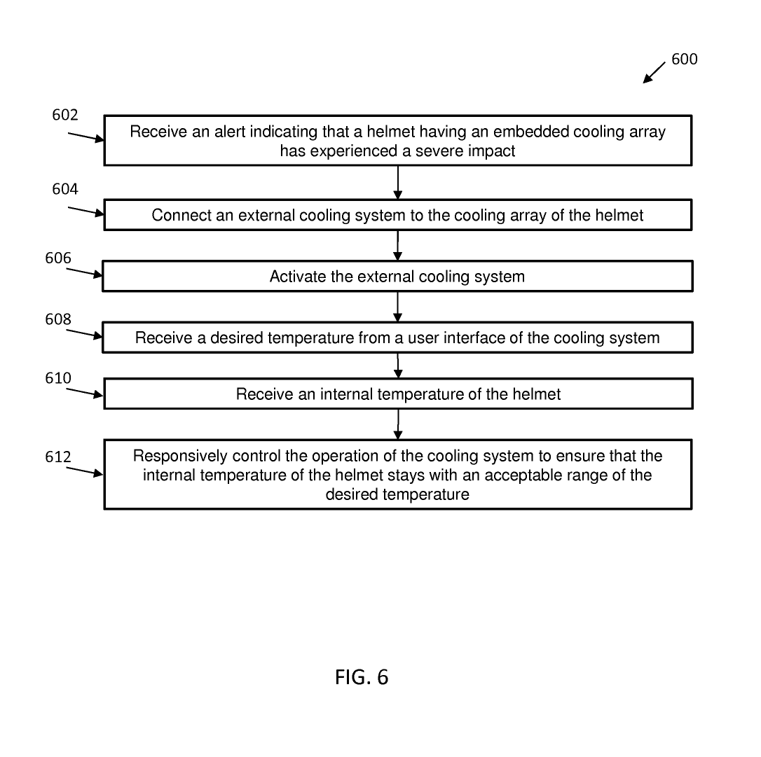

Referring now to FIG. 6, a flow diagram of a method 600 for cooling a helmet having an embedded cooling array with an external cooling system in accordance with an exemplary embodiment is shown. As shown at block 602, the method 600 includes receiving an alert indicating that the helmet has experienced a severe impact. Next, as shown at block 604, the method 600 includes connecting the external cooling system to the cooling array of the helmet The method 600 also includes activating the external cooling system, as shown at block 606. Next, as shown at block 604, the method 600 includes receiving a desired temperature from a user interface of the cooling system. The method 600 also includes receiving an internal temperature of the helmet, as shown at block 606. Next, as shown at block 604, the method 600 includes responsively controlling the operation of the cooling system to ensure that the internal temperature of the helmet stays with an acceptable range of the desired temperature.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for cooling a helmet having an embedded cooling array with an external cooling system, the method comprising:
determining that the helmet has experienced a severe impact, wherein the helmet includes a padding configured to protect a user of the helmet from impacts;
connecting the external cooling system to the embedded cooling array of the helmet; and
activating the external cooling system, wherein activating the external cooling system decreases an internal temperature of the helmet via the embedded cooling array.

2. The method of claim 1, further comprising:
receiving a desired temperature from a user interface of the external cooling system;
receiving the internal temperature of the helmet from a thermometer disposed within the helmet; and
responsively operating the external cooling system to ensure that the internal temperature of the helmet stays with an acceptable range of the desired temperature.

3. The method of claim 1, wherein the helmet includes one or more cooling system ports that are accessible when the helmet is disposed on the user and which are configured to connect the embedded cooling array to the external cooling system.

4. The method of claim 1, wherein the embedded cooling array includes one or more tubes for circulating cooling fluid.

5. The method of claim 4, wherein the one or more tubes for circulating cooling fluid are disposed within the padding.

6. The method of claim 1, wherein determining that the helmet has experienced the severe impact includes receiving an alert from a transceiver disposed within the helmet.

7. The method of claim 1, wherein the embedded cooling array includes one or more thermoelectric cooling pads and wherein the external cooling system includes an electrical system designed to selectively activate the one or more thermoelectric cooling pads.

8. The method of claim 1, wherein activating the external cooling system includes activating only a portion of the embedded cooling array based on an assessment of the cognitive function of the user of the helmet performed by a medical professional.
